(19) United States
(12) Patent Application Publication
(10) Pub. No.: US 2023/0039443 A1
(43) Pub. Date: Feb. 9, 2023

(54) PROCESS FOR BUILDING NANOPARTICLE-BASED DRUG CARRIERS VIA PROTEIN CORONA MODULATION

(71) Applicant: NANOPEPTIDE (QINGDAO) BIOTECHNOLOGY LTD., Qingdao, Shandong (CN)

(72) Inventors: Sajeev KOHLI, Qingdao, Shandong (CN); Alireza Zehtab YAZDI, Qingdao, Shandong (CN); Pu CHEN, Qingdao, Shandong (CN)

(21) Appl. No.: 17/759,046

(22) PCT Filed: Jan. 20, 2020
(86) PCT No.: PCT/CN2020/073273

§ 371 Date: Jul. 19, 2022

(51) Int. Cl. A61K 47/69; C12N 15/113; A61K 47/54; A61K 47/68; G01N 33/68; A61P 35/00

(52) U.S. Cl. CPC A61K 47/6929; C12N 15/1135; A61K 47/549; A61K 47/6851; A61K 47/6843; G01N 33/6845; A61P 35/00; C12N 2320/32; C12N 2310/14; C12N 2310/3513

(57) ABSTRACT

The invention relates to the method for building nanoparticle-based drug carriers and the nanoparticle based drug delivery system able to manipulate the corresponding protein corona for specific and potent drug delivery to cancer cells.

### FIELD OF THE INVENTION

The invention relates to the field of biotechnology, in particular, to the method for building nanoparticle-based drug carriers and the nanoparticle based drug delivery system able to manipulate the corresponding protein corona for specific and potent drug delivery to cancer cells.

### BACKGROUND OF THE INVENTION

Over the past decade, nanotechnology has offered immense promise for biomedical applications, allowing for therapeutics to be engineered for remediating a wide variety of diseases that maintain the benefits of prolonged half-life, improved bio-distribution, increased circulation time and many other benefits. However, many nanotechnology-based therapeutics that should theoretically work well in concept do not work nearly as well in practice. This is mainly because targeting transformed cells, or getting the therapeutic to where it needs to go, is a very challenging task.

One of the main reasons for the lack of success of nanoparticle based therapeutics is called the protein corona. When a drug enters the body intravenously, the first physiological compartment it contacts is the blood. The blood contains an abundance of thousands of proteins, a large subset of which adsorb onto the drug and change essentially all of its synthetic properties including size, dispersion, aggregation state, bio-targeting ability. These proteins give the drug a whole new biological identity differing from its initial synthetic identity and it is the biological identity that the cell actually ends up seeing. Uncontrolled protein nanoparticle interactions impede the drug from going to where it is supposed to go, resulting in lack of success of the therapeutic.

Another reason for lack of success of nanoparticle based therapeutics involves the fundamental methodology associated with how one goes about making a drug carrier. A nano-therapeutic consists of three main components: a base, a sensor molecule, and a payload. Researchers often just mix and match based on theoretical knowledge as to what should be the best configuration. There is no real systematic or deterministic way of knowing for sure that this will be the best configuration. In most cases, this results in a large amount of time, money, and resources being wasted on configurations that do not work as well as initially anticipated.

Combining the issues of uncontrolled adsorption of proteins following exposure to a physiological system in addition to non-systematic approaches for drug carrier development makes it clear to see why nanotechnology-based therapeutics are so far from widespread implementation. Certain studies have tried using techniques such as PEGylation and zwitterionic nanoparticles to impede the adsorption of proteins in the protein corona, in an attempt to mitigate it completely; however even with this slight “masking effect”, there is still some protein adsorption that occurs on the carrier surface, enough to allow the corona layer associated with the “biological identity” to form nonetheless, resulting in the synthetic identity of the nanoparticle being masked. In addition, until now, other than using theoretical knowledge to anticipate ideal carrier configurations, there has not been any attempt to develop a systematic process for building nanoparticle based drug carriers to ensure the most ideal result.

The proposed invention addresses both of the aforementioned issues. Through combining high-throughput shotgun-based proteomics on the nano-scale and bioinformatics, the proposed process enables one to build nanoparticle-based drug carriers that are able to use the protein corona as a strength instead of a weakness, controlling nanoparticle-protein interactions in a systematic manner to allow the protein corona to transport the resulting complexes to their intended biological sites. Initially, the properties of the protein coronae on a library of graphene or graphene oxide derivatives with differing physicochemical properties was assessed. Subsequently, a novel data mining algorithm was used for finding possible proteins to recruit onto the corona of each graphene derivative to target specified cells at designated biological sites. Finally, nucleic acid-graphene oxide-antibody complexes were engineered for recognizing these target protein(s) and harnessing their physiological carrier functionalities, which were then evaluated for their performance in gene transfection, cell viability and cellular uptake in vitro. Overall, an entirely novel workflow for gene carrier development was implemented and evaluated for overall effectiveness. The proposed workflow is universal in its application, as it can be applied to any nanoparticle library. This novel, streamlined process takes 4 days to complete and is relatively inexpensive, where the resulting carriers outperform conventional gold standards for intracellular drug delivery.

### SUMMARY OF THE INVENTION

The present invention consists of a process for building nanoparticle-based drug carriers that are able to harness the physiological carrier functionalities of endogenous proteins, i.e. proteins native to human serum, for site-specific targeting of cancer cells. The process consists of initially exposing constituents of a synthetic nanoparticle library to 10% human serum in PBS, followed by a 90-minute incubation period at 37 degrees Celsius, followed by repeated centrifugation for isolation and washing of the corona layer. Following protein corona isolation, each corona isolate is analyzed using liquid chromatography tandem mass spectrometry on the nano-scale (nano LC-MS) for profiling the identity and quantity of the adsorbed proteins on each nanoparticle formulation. The proteins corresponding to each nanoparticle formulation are then inputted into a computer algorithm in which a novel bioinformatic screening strategy is used to rank the hundreds of proteins that adsorb to each nanoparticle formulation based on their ability to be recognized by cell receptors overexpressed in cancer cells. An antibody against the outputted “best” corona protein is then functionalized onto the outputted “best” nanoparticle formulation via conventional EDC-NHS crosslinking. A therapeutic payload, consisting of siRNA against the BCl2 oncogene, is then adsorbed onto the nanoparticle formulation via a simple mixing process via passive adsorption. The resulting conjugate is now able to recruit helpful endogenous proteins into the corona layer that encourage site-specific targeting of cancer cells, turning the associated protein corona into a strength instead of a weakness. The process itself takes four days for completion and can be applied to any nanoparticle type and any therapeutic payload given the mechanism of interaction between the payload and nanoparticle is that of passive adsorption. A wide range of diseases may also be targeted depending on the inputted parameters in the engineered bioinformatic algorithm.

Specifically, in one aspect, the present invention relates to a method for building nanoparticle-based drug carriers for the controlled, intracellular administration of drugs by manipulation of the nanoparticle protein corona through a combination of: A. liquid chromatography tandem mass spectrometry on the nano-scale of corona extracts prepared from nanoparticle formulations; B. high throughput data mining for determining tens of thousands of protein-protein interactions associated with said corona extracts to then determine which of said corona proteins are most ideal to recruit endogenously for increasing likelihood of cell specific uptake; C. antibody conjugation, where antibodies against said ideal corona protein are determined by said algorithm; D. Incorporating the drug into said nanoparticle-antibody conjugate.

In one embodiment, wherein said drugs consist of siRNA therapeutics.

In one embodiment, wherein said siRNA is against the BCL-2 oncogene.

In one embodiment, wherein said nanoparticle formulations consist of derivatives of graphene or graphene oxides

In one embodiment, wherein said high throughput data mining is attained by a combination of Python Scripts mining through existing Gene Ontology, Protein-Protein Interaction, and mRNA transcriptomic databases, writing to a master My SQL database.

In one embodiment, wherein said antibodies consist of monoclonal antibodies.

In one embodiment, wherein said cells correspond to cancer cells.

In another aspect, provided herein is a method for building nanoparticle-based drug carriers for the controlled, intracellular administration of drugs by manipulation of the nanoparticle protein corona through a combination of: A. exposing nanoparticle formulations to human serum with normalized surface area (of the nanoparticles) to volume (culture volume) ratios over a set incubation time followed by corona isolation for nano LC-MS/MS analysis; B. deploying a series of scripts over a series of pre-existing proteomics data repositories for distinguishing ideal corona proteins for endogenous recruitment; C. Employing EDC-NHS crosslinking for conjugating antibodies against the ideal corona protein to the ideal nanoparticle formulation; D. Employing passive adsorption to conjugate siRNA to the nanoparticle surface.

In one embodiment, wherein said surface area to volume ratio is set to 1 to 10 cm2/uL.

In one embodiment, wherein exposure time to human serum is 1-2 hours.

In another aspect, provided herein is a nanoparticle based drug delivery system able to manipulate the corresponding protein corona for specific and potent drug delivery to cancer cells comprising a combination of: A. a series of monoclonal antibodies tethered to said nanoparticle surface to increase the abundance of a particular protein in the corona for cancer-cell specific uptake and B. a series of polymers with ethyl and oxide functionalities to enhance solubility.

In one embodiment, wherein said monoclonal antibodies are against the human serotransferrin protein.

In one embodiment, wherein said monoclonal antibodies are conjugated to yield a final concentration of 25-50 ug/mL.

### BRIEF DESCRIPTION OF THE DRAWINGS

These and other aspects and advantages of embodiments of the present disclosure will become apparent and more readily appreciated from the following descriptions made with reference the accompanying schemes and drawings, in which:

FIG. 1 shows example Protein-Protein Interaction Network for Serotransferrin (a single corona protein)—made with Cytoscape.

FIG. 2 shows high throughput mRNA Transcriptome Analysis Results for nGO 200 Corona Isolate—5 000 corona protein-receptor pairs ranked on differential expression values in lung cancer. Serotransferrin (and the associated transferrin receptor) were ranked as number 1.

FIG. 3 shows quantitative comparison of intracellular localization of GO-Ab vs. GO-Tf conjugates. The blue line represents GO-Ab conjugates and the red represents GO-Tf.

FIG. 4 shows confocal microscope images allowing visualization of uptake of GO-Tf vs. GO-Ab conjugates in human serum environment. Green represents FITC reporter (GO-Tf or GO-Ab), blue represents DAPI (cell nuclei) and red is associated with Rhodamine Phalloidin stain (cell membrane).

FIG. 5 shows representation of extracted ion currents from peptides identified to be associated with serotransferrin. The above figure corresponds to the sequence IECVSAETTEDCIAK. Other sequences analyzed for comparison of serotransferrin abundance in GO vs. GO-Ab conjugates include ASYLDCIR, EDPQTFYYAVAVVK, and DCHLAQVP.

FIG. 6 shows representation of BCl2 knockdown efficiencies of GO, GO-Tf, lipofectamine 2000, and GO-Anti-Tf coupled siRNA complexes in lung cancer cells.

### DETAILED DESCRIPTION OF THE INVENTION

The present invention consists of a novel, multi-step methodology that can be used for building nanoparticle-based drug carriers able to control protein corona formation for increasing targeting ability to certain cell populations. As such, in describing the invention, each individual step will be elaborated upon in detail.

The initial step consists of subjecting nanoparticle formulations to 10% human serum in phosphate buffer saline. Initially, graphene oxide nanoparticles are synthesized via chemical exfoliation method from stock graphite nanoplatelets. The dry nanoparticles are dissolved in MiliQ water at a concentration of 1 mg/mL through a combination of vigorous vortexing and probe sonication. Following the generation of a stable suspension, a table containing the characteristics of the nanoparticles, obtained through atomic force microscopy, is used to perform mathematical calculations to ensure that each nanoparticle formulation is subjected to the same volume (of human serum) to surface area (of nanoparticle formulations) ratio. The results of the calculations allow one to determine the specific volume of graphene solution to take from the prepared stock and transfer to the 10% human serum in PBS.

Following exposure to serum solutions, the nanoparticles are incubated at 37 degrees Celsius for 2 hours, followed by centrifugation at 16 000 rpm at 4 degrees Celsius for half an hour and re-suspension in PBS EDTA. Three washing steps of this sort are taken, followed by removal of the supernatant to the extent that only 15 ul of liquid remain in each sample. Samples are then subjected to DTT and 10% SDS, followed by incubation for 1 hour at 70 degrees Celsius. Samples are centrifuged at 16 000 rpm at 4 degrees Celsius for half an hour and re-suspended in 10% TCA in acetone, followed by overnight incubation at −80 degrees Celsius.

The protein isolates are then centrifuged at 16 000 g for 30 minutes at 4° C., followed by addition of 500 uL 0.05% sodium deoxycholate and 100 uL 72% TCA. They are subsequently incubated on ice for 30 minutes, followed by centrifugation at 16,000 g for 30 minutes at 4° C., and resuspension in 1 mL acetone. Protein isolates were washed in acetone for 1 hour, after which pellets were dried in a fume hood and redissolved in 50 mM ammonium bicarbonate.

The resulting corona isolates are then taken for analysis via liquid chromatography tandem mass spectrometry on the nano-scale, using a C18 reverse phase liquid chromatography column. Samples are alkylated to remove cysteine residues and exposed to trypsin to break proteins down into peptides for ease of LC-MS Analysis. Scaffold is used to analyze nano-LC MS data, resulting in lists of hundreds of proteins per nanoparticle formulation. The corresponding lists are then exported into excel for subsequent analysis through the bioionformatic screening process.

A brief explanation of the algorithm follows. After looking at the physiological functionalities of hundreds of proteins for each nanoparticle corona extract through gene ontology screening, protein-protein interaction databases can be used to screen tens of thousands of potential interactions in which these corona proteins can participate. The resulting list of protein interactors can then be pruned down to only include those proteins with a cell surface receptor functionality. The final list of cell surface receptors able to recognize and or internalize inputted corona proteins can then be subjected to high throughput mRNA transcriptome analysis over thousands of cell lines to rank them based on differential expression values. The number one protein on the resulting list of thousands of corona protein-cell receptor pairs is finally recognized as the most suitable protein for endogenous recruitment.

The algorithm itself consists of four main steps. The nature of each step and results are explained in the following sections in detail.

Gene Ontology Profiling

As an initial step, a Python Script (attached at the end of this filing) is deployed to search all proteins identified in each individual nanoparticle protein corona extract against the QuickGO database to identify the corresponding physiological functions. This results in the creation of a table as part of a MySQL database, with parameters such as a unique interactor ID in the form of a UniProtKB ID corresponding to each corona protein, a Gene Ontology (GO) Class Number corresponding to a particular physiological function associated with that corona protein, and a text label denoting the physiological function enumerated by the GO Class number. Certain proteins are associated with tens of thousands of functions, while others were associated with hundreds. In all, this step usually results in the identification of 100 000 physiological functions per corona isolate.

Protein-Protein Interaction Screening

The corona proteins are then searched against over twenty different protein-protein interaction databases (APID Interactomes, BindingDB, DIP-IMEx, GeneMANIA, InnateDB, iRefIndex, MINT, Spike, ZINC, BAR, BioGrid, DrugBank, HPIDb, InnateDB-All, Matrix DB, MPIDB, ChEMBL, EBI-GOA-miRNA, I2D, IntAct, MBlnfo, Reactome, UniProt, BIND, DIP, EBI-GOA-non-IntAct, mentha) using the iRefIndexAggregator virtual toolbox. This results in the creation of another table as part of a MySQL database, with tables corresponding to gene or protein symbol corresponding to a particular corona protein, a unique identifier associated with the database at hand for that corona protein, a particular interactor symbol for a protein that can interact with the inputted corona protein, and a unique identifier associated with the database at hand for this interactor protein. Over 40 000 proteins are usually identified to be able to interact with each corona isolate.

Receptor Pruning

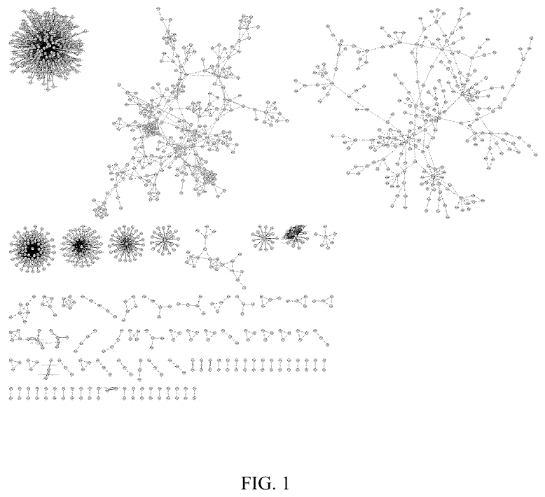

The outputted interactor proteins are then searched against QuickGO again and only those corresponding to a receptor functionality based GO Class term re returned, corresponding to cell surface receptors. In an initial list of 40 000 corona proteins, hundreds of thousands of GO Class terms are filtered down to only include classes and subclasses corresponding to 127 terms, relating to cell surface receptor functionality. The result is shown in FIG. 1.

High Throughput mRNA Transcriptome Analysis

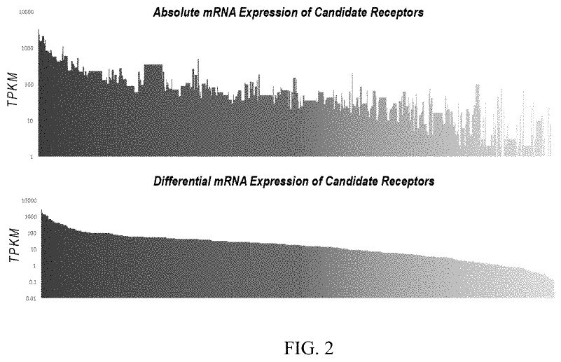

The resulting cell surface receptors are then subjected to high throughput mRNA transcriptome analysis and ranked based on differential expression in a target cell population (as shown in FIG. 2). Expression values for each receptor from the results of the receptor pruning process were analyzed over hundreds of cell lines corresponding to lung cancer cells as an initial model and compared to expression values associated with normal cells in transcripts per million (TPKM) in terms of mRNA expression. These normal expression values were subtracted from the cancerous expression values to determine differential expression values in transcripts per million for each receptor. Receptors are finally ranked based on these differential receptor expression values, resulting in around 5000 potential candidates per corona extract (for lung cancer, breast cancer, and colorectal cancer individually).

The number 1 corona protein is now known to be internalized by cell receptors overexpressed in the target cell population. Increasing its abundance in the corona would thus increase the probability of internalization by this cell population. To increase the abundance of the corona protein, synthetic nanoparticles found to have the highest amount of this protein in the corresponding corona are functionalized with monoclonal antibodies against this corona protein via simple EDC NHS crosslinking. The resulting conjugates are filtered via centrifugal filtration columns. siRNA against BCl2 can now be functionalized onto the resulting conjugates via simple passive adsorption in an ice bath, subsequently stirred for 2 hours, and centrifuged to obtain the resulting carrier.

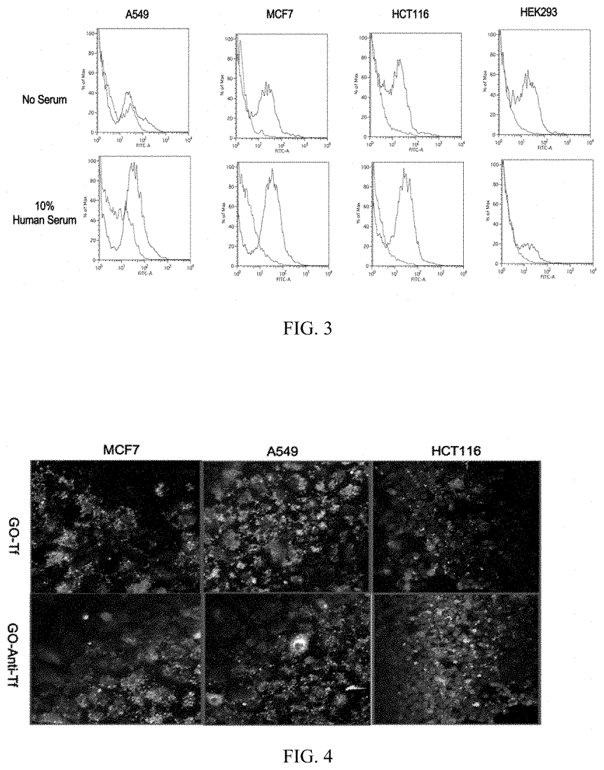

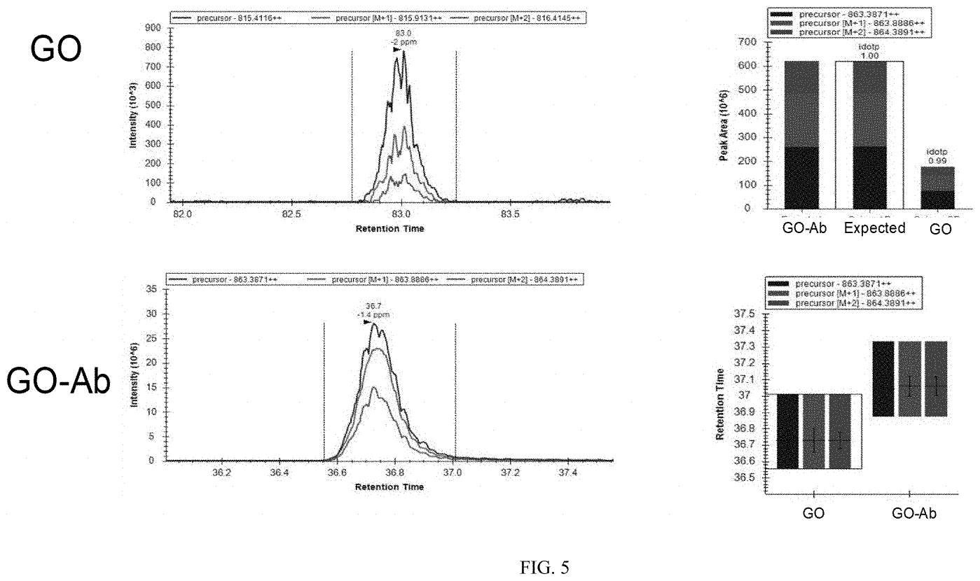

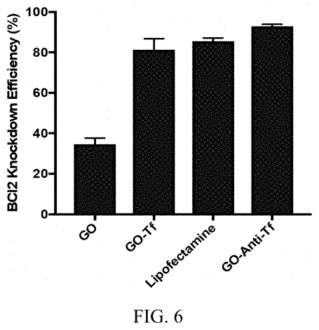

The conjugates in solution are placed in an ice bath, followed by exposure of siRNA and subsequent stirring on ice for 2 hours. The conjugates are centrifuged to separate the resulting complexes, which can then be immediately used for transfection purposes. The advantages of the resulting conjugate include low cost, as graphene oxide is almost 100 times less expensive than lipofectamine, the gold standard of siRNA transfection, in addition to ease of preparation. The total yield of siRNA adsorbed onto the graphene surface compared to that initially exposed to the conjugate is well over 90% as a result of the high surface area to volume ratio of the corresponding nanoparticle formulation. LC-MS and Flow Cytometry experiments confirm the ability of the graphene oxide-anti-transferrin monoclonal antibody-BCl2 siRNA complexes to recruit up to 2-3 times more transferrin than a graphene oxide formulation that has not been functionalized with anti-transferrin antibodies. The result is shown in FIG. 3. The conjugates also exhibit a significantly higher internalization than the non-functionalized counterparts in lung, breast, and colorectal cancer cells (as shown in FIG. 4) and notably, minimal to no internalization when the transferrin receptor has been pre-blocked, (as shown in FIG. 5) concluding that the internalization is indeed transferrin receptor assisted. The indirect targeting approach employed in the construction of this nanoparticle based drug carrier is the first of its kind (as shown in FIG. 6).

1. A method for building nanoparticle-based drug carriers for controlled, intracellular administration of drugs by manipulation of nanoparticle protein corona through a combination of:
A. liquid chromatography tandem mass spectrometry on a nano-scale of corona extracts prepared from nanoparticle formulations;
B. high throughput data mining for determining tens of thousands of protein-protein interactions associated with said corona extracts to then determine which of said corona proteins are most ideal to recruit endogenously for increasing likelihood of cell specific uptake;
C. antibody conjugation, where antibodies against said ideal corona protein are determined by an algorithm; and
D. incorporating the drug into said nanoparticle-antibody conjugate.

2. The method of claim 1, wherein said drugs consist of siRNA therapeutics.

3. The method of claim 2, wherein said siRNA is against BCL-2 oncogene.

4. The method of claim 1, wherein said nanoparticle formulations consist of derivatives of graphene or graphene oxides.

5. The method of claim 1, wherein said high throughput data mining is attained by a combination of Python Scripts mining through existing Gene Ontology, Protein-Protein Interaction, and mRNA transcriptomic databases, writing to a master MySQL database.

6. The method of claim 1, wherein said antibodies consist of monoclonal antibodies.

7. The method of claim 1, wherein said cells correspond to cancer cells.

8. A method for building nanoparticle-based drug carriers for controlled, intracellular administration of drugs by manipulation of nanoparticle protein corona through a combination of:
A. exposing nanoparticle formulations to human serum with normalized surface area (of the nanoparticles) to volume (culture volume) ratios over a set incubation time followed by corona isolation for nano LC-MS/MS analysis;
B. deploying a series of scripts over a series of pre-existing proteomics data repositories for distinguishing ideal corona proteins for endogenous recruitment;
C. employing EDC-NHS crosslinking for conjugating antibodies against an ideal corona protein to an ideal nanoparticle formulation; and
D. employing passive adsorption to conjugate siRNA to the nanoparticle surface.

9. The method of claim 8, wherein said surface area to volume ratio is set to 1 to 10 cm²/uL.

10. The method of claim 8, wherein exposure time to human serum is 1-2 hours.

11. A nanoparticle based drug delivery system able to manipulate a corresponding protein corona for specific and potent drug delivery to cancer cells comprising a combination of:
A. a series of monoclonal antibodies tethered to a nanoparticle surface to increase an abundance of a particular protein in the corona for cancer-cell specific uptake; and
B. a series of polymers with ethyl and oxide functionalities to enhance solubility.

12. The nanoparticle based drug delivery system of claim 11, wherein said monoclonal antibodies are against human serotransferrin protein.

13. The nanoparticle based drug delivery system of claim 11, wherein said monoclonal antibodies are conjugated to yield a final concentration of 25-50 ug/mL.
